(19) United States
(12) Patent Application Publication
(10) Pub. No.: US 2026/0026723 A1
(43) Pub. Date: Jan. 29, 2026

(54) HEALTH STATE EVALUATION APPARATUS, HEALTH STATE EVALUATION METHOD, AND STORAGE MEDIUM

(71) Applicant: HONDA MOTOR CO., LTD., Tokyo (JP)

(72) Inventors: Satoru Shinkawa, Wako-shi (JP); Shigenobu Mitsuzawa, Wako-shi (JP); Hiroshi Ono, Wako-shi (JP); Hiroki Taniguchi, Wako-shi (JP); Nozomi Nagatani, Wako-shi (JP)

(21) Appl. No.: 19/211,654

(22) Filed: May 19, 2025

(30) Foreign Application Priority Data
Jul. 26, 2024 (JP) 2024-121012

(51) Int. Cl. A61B 5/18; A61B 5/00; G07C 5/02; G16H 50/30

(52) U.S. Cl. CPC A61B 5/18; A61B 5/0002; A61B 5/681; G07C 5/02; G16H 50/30

(57) ABSTRACT

A smartphone includes: an acquisition unit configured to acquire first biological information from a smartwatch that is worn by a user and detects biological information about the user, and acquire second biological information from an information holding institution that holds biological information about the user; and an evaluation unit configured to evaluate whether a health state of the user is an appropriate state as a driver of a vehicle, based on the first biological information and the second biological information.

### INCORPORATION BY REFERENCE

The present application claims priority under 35 U.S.C. § 119 to Japanese Patent Application No. 2024-121012 filed on Jul. 26, 2024. The content of the application is incorporated herein by reference in its entirety.

### BACKGROUND OF THE INVENTION

### Field of the Invention

The present invention relates to a health state evaluation apparatus, a health state evaluation method, and a storage medium.

### Description of the Related Art

There has been proposed an apparatus that acquires biological information about a driver and driving information relating to a driving operation of the driver, and determines appropriateness of the driver's driving based on these acquired pieces of information (for example, see Japanese Patent Laid-Open No. 2023-145468).

In recent years, efforts have gained momentum for providing access to sustainable transport systems that also take into account people in vulnerable situations among traffic participants. Towards this end, efforts are focused on research and development for further improving traffic safety, convenience, and the like through research and development relating to a driver assistance system that supports the safety and health of drivers.

However, in the driver assistance system, a problem is to further improve traffic safety, convenience, and the like by appropriately evaluating a health state related to a driving ability of a driver.

In order to solve the above-described problem, an object of the present application is to appropriately evaluate a health state related to a driving ability of a driver. Thus, the present application contributes to development of a sustainable transport system.

### SUMMARY OF THE INVENTION

According to a first aspect for achieving the above object, a health state evaluation apparatus includes: an acquisition unit configured to acquire first biological information from a device that is worn by a user and detects biological information about the user, and acquire second biological information from an information holding institution that holds biological information about the user; and an evaluation unit configured to evaluate whether a health state of the user is an appropriate state as a driver of a vehicle, based on the first biological information and the second biological information.

As a second aspect for achieving the above object, a health state evaluation method includes: an acquisition step of acquiring first biological information from a device that is worn by a user and detects biological information about the user, and acquiring second biological information from an information holding institution that holds biological information about the user; and an evaluation step of evaluating whether a health state of the user is an appropriate state as a driver of a vehicle, based on the first biological information and the second biological information.

According to a third aspect for achieving the above object, a non-transitory computer readable storage medium storing a health state evaluation program causes a processor included in a health state evaluation apparatus to function as: an acquisition unit configured to acquire first biological information from a device that is worn by a user and detects biological information about the user, and acquire second biological information from an information holding institution that holds biological information about the user; and an evaluation unit configured to evaluate whether a health state of the user is an appropriate state as a driver of a vehicle, based on the first biological information and the second biological information.

According to the health state evaluation apparatus, the health state evaluation method, and the storage medium described above, it is possible to appropriately evaluate a health state related to a driving ability of a user.

### BRIEF DESCRIPTION OF THE DRAWINGS

FIG. 1 is a diagram showing an example of a configuration of a health state evaluation system;

FIG. 2 is a diagram showing an example of a configuration of a smartwatch;

FIG. 3 is a diagram showing an example of a configuration of an in-vehicle apparatus;

FIG. 4 is a diagram showing an example of a configuration of a smartphone;

FIG. 5 is a diagram showing an example of a correlation between biological information, a rating, and advice information; and

FIG. 6 is a flowchart showing an example of processing of the smartphone.

### DETAILED DESCRIPTION OF THE INVENTION

Hereinafter, embodiments of a health state evaluation apparatus, a health state evaluation method, and a health state evaluation program will be described with reference to the drawings.

### 1. Configuration of Health State Evaluation System

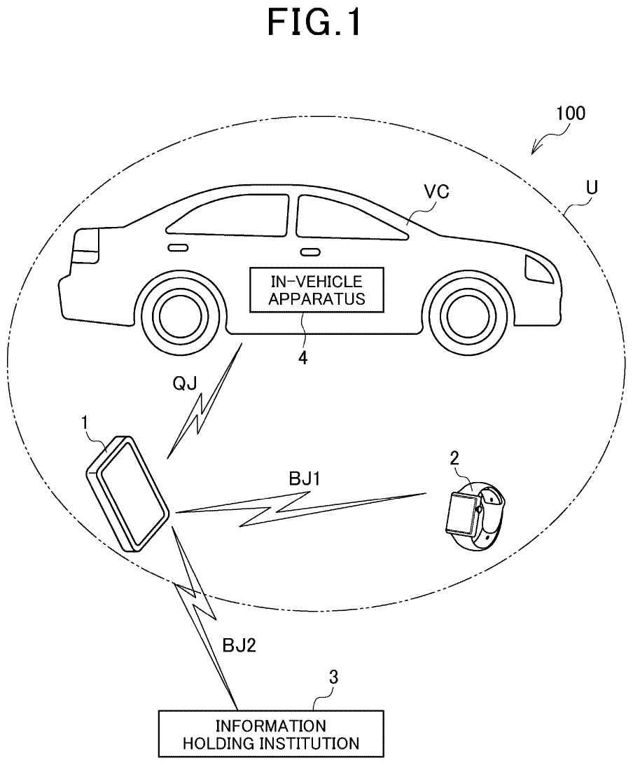

First, a configuration of a health state evaluation system 100 will be described with reference to FIG. 1. FIG. 1 is a diagram showing an example of the configuration of the health state evaluation system 100. The health state evaluation system 100 corresponds to an example of the above “driver assistance system”.

As shown in FIG. 1, the health state evaluation system 100 includes a smartphone 1, a smartwatch 2, and an in-vehicle apparatus 4. Note that a dash-dotted line in FIG. 1 indicates that the smartphone 1, the smartwatch 2, and a vehicle VC are property of a user U.

The smartphone 1 wirelessly communicates with the smartwatch 2, an information holding institution 3, and the in-vehicle apparatus 4 in accordance with, for example, a communication standard such as Wi-Fi®.

The smartphone 1 acquires first biological information BJ1 from the smartwatch 2. The user U is a driver that drives the vehicle VC. The smartphone 1 acquires second biological information BJ2 from the information holding institution 3. The smartphone 1 acquires driving information QJ from the in-vehicle apparatus 4.

The smartphone 1 evaluates whether a health state of the user U is an appropriate state as the driver of the vehicle VC, based on the first biological information BJ1 and the second biological information BJ2.

The smartphone 1 corresponds to an example of the “health state evaluation apparatus”.

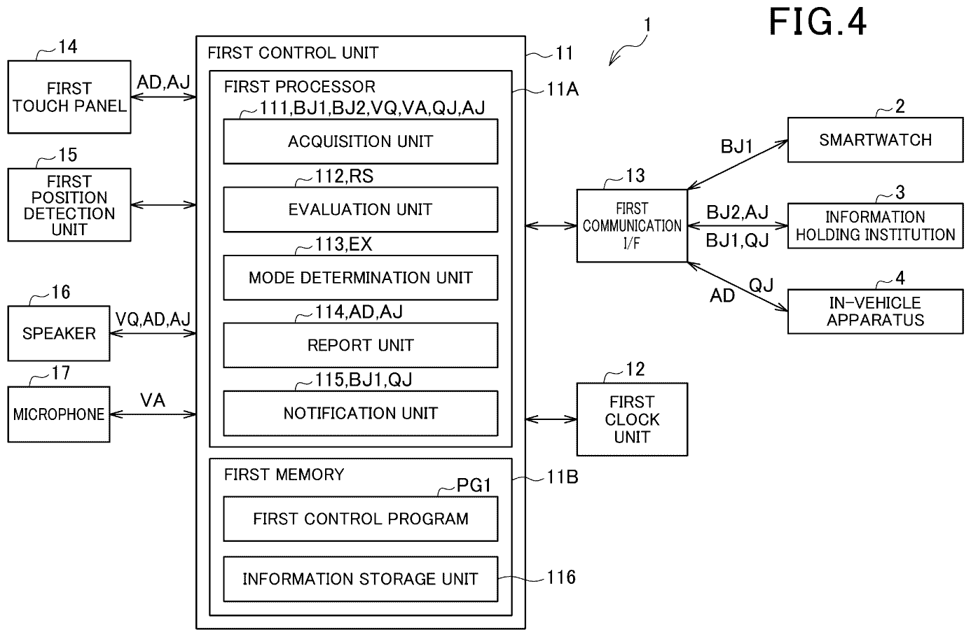

The smartphone 1 will be described further with reference to FIGS. 4 to 6.

The smartwatch 2 is worn on a wrist of the user U. The smartwatch 2 generates the first biological information BJ1. The first biological information BJ1 includes, for example, a heart rate of the user U.

The smartwatch 2 corresponds to an example of a “device”.

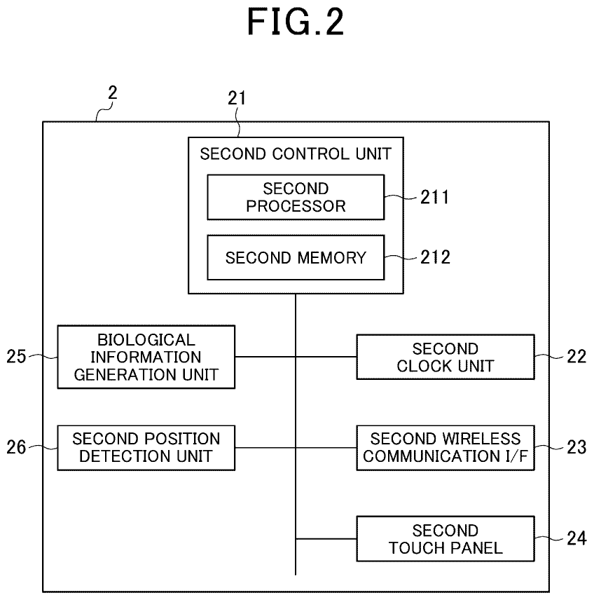

The smartwatch 2 will be described further with reference to FIG. 2.

The information holding institution 3 is an institution that holds the second biological information BJ2. The second biological information BJ2 includes, for example, health diagnosis information about the user U. The information holding institution 3 generates the second biological information BJ2. The information holding institution 3 is an institution that holds the second biological information BJ2, such as a hospital, an insurance company, a municipality, or a caregiving company. In the present embodiment, the information holding institution 3 indicates, for example, a server apparatus disposed in a hospital or the like.

The second biological information BJ2 will be described further with reference to FIGS. 4 to 6.

The in-vehicle apparatus 4 is installed in the vehicle VC. The in-vehicle apparatus 4 generates the driving information QJ. The driving information QJ includes operation information from when the user U has driven the vehicle VC.

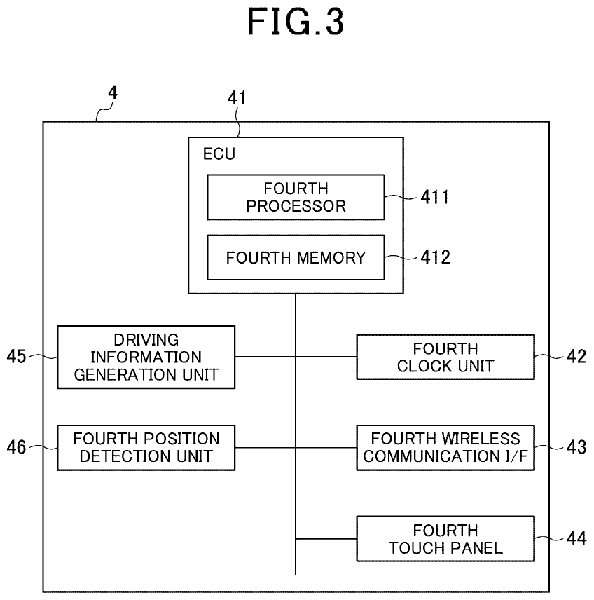

The in-vehicle apparatus 4 will be described further with reference to FIG. 3.

### 2. Configuration of Smartwatch

Next, a configuration of the smartwatch 2 will be described with reference to FIG. 2. FIG. 2 is a diagram showing an example of the configuration of the smartwatch 2.

As shown in FIG. 2, the smartwatch 2 includes a second control unit 21, a second clock unit 22, a second wireless communication interface 23, a second touch panel 24, a biological information generation unit 25, and a second position detection unit 26.

The second control unit 21 controls each unit of the smartwatch 2. The second control unit 21 includes a second processor 211 and a second memory 212.

The second memory 212 consists of, for example, a non-volatile semiconductor memory, or volatile and non-volatile semiconductor memories. The second processor 211 consists of a central processing unit (CPU), and microprocessor unit (MPU), or the like. The second processor 211 may consist of a single processor or can also consist of a plurality of processors.

The second processor 211 controls each unit of the smartwatch 2 by executing a second control program stored in the second memory 212.

The second clock unit 22 has a clock function of measuring a date and time. The second clock unit 22 acquires date and time information from a server apparatus (not illustrated). The second clock unit 22 adjusts the clock function by using the acquired date and time information. The second clock unit 22 displays the date and time information on the second touch panel 24.

The second wireless communication interface 23 includes an antenna that receives a wireless signal and an interface circuit that processes the wireless signal received by the antenna. The second wireless communication interface 23 performs wireless communication with external apparatuses including the server apparatus (not illustrated) and the smartphone 1.

The second touch panel 24 includes a display panel and a touch sensor. The display panel consists of a liquid crystal panel, an organic electroluminescent (EL) panel, or the like. The touch sensor detects a touch operation of the user U on the display panel.

The second touch panel 24 displays an image on the display panel in accordance with an instruction from the second control unit 21. The second touch panel 24 detects a touch operation of the user U on the display panel.

The biological information generation unit 25 is a computer (ECU) including a processor and a memory, and generates the first biological information BJ1.

The biological information generation unit 25 measures, at preset intervals, the first biological information BJ1 that is the biological information about the user U who is wearing the smartwatch 2.

The first biological information BJ1 includes, for example, sleep information BJ11, exercise information BJ12, and stress information BJ13.

The sleep information BJ11, the exercise information BJ12, the stress information BJ13 will be described further with reference to FIG. 4.

The biological information generation unit 25 outputs the measured first biological information BJ1 to the second control unit 21. The second control unit 21 transmits the first biological information BJ1 to the smartphone 1 via the second wireless communication interface 23.

The second position detection unit 26 includes a global navigation satellite system (GNSS) sensor. The second position detection unit 26 measures a position, that is, a latitude, a longitude, an altitude, and the like of the smartwatch 2, by receiving a positioning signal transmitted from a GNSS satellite.

The second position detection unit 26 outputs position information indicating the measured position to the second control unit 21.

### 3. Configuration of In-Vehicle Apparatus

Next, a configuration of the in-vehicle apparatus 4 will be described with reference to FIG. 3. FIG. 3 is a diagram showing an example of the configuration of the in-vehicle apparatus 4.

As shown in FIG. 3, the in-vehicle apparatus 4 includes an electronic control unit (ECU) 41, a fourth clock unit 42, a fourth wireless communication interface 43, a fourth touch panel 44, a driving information generation unit 45, and a fourth position detection unit 46.

The ECU 41 controls each unit of the in-vehicle apparatus 4. The ECU 41 includes a fourth processor 411 and a fourth memory 412.

The fourth memory 412 consists of, for example, a non-volatile semiconductor memory, volatile and non-volatile semiconductor memories, or the like. The fourth processor 411 consists of a CPU, an MPU, or the like. The fourth processor 411 may consist of a single processor or can also consist of a plurality of processors.

The fourth processor 411 controls each unit of the in-vehicle apparatus 4 by executing a fourth control program stored in the fourth memory 412.

The fourth clock unit 42 has a clock function of measuring the date and time. The fourth clock unit 42 acquires the date and time information from the server apparatus (not illustrated). The fourth clock unit 42 adjusts the clock function by using the acquired date and time information. The fourth clock unit 42 displays the date and time information on the fourth touch panel 44.

The fourth wireless communication interface 43 includes an antenna that receives a wireless signal and an interface circuit that processes the wireless signal received by the antenna. The fourth wireless communication interface 43 performs wireless communication with external apparatuses including the server apparatus (not illustrated) and the smartphone 1.

The fourth touch panel 44 includes a display panel and a touch sensor. The display panel consists of a liquid crystal panel, an organic EL panel, or the like. The touch sensor detects a touch operation of the user U on the display panel.

The fourth touch panel 44 displays an image on the display panel in accordance with an instruction from the ECU 41. The fourth touch panel 44 detects a touch operation of the user U on the display panel.

The driving information generation unit 45 is a computer (ECU) including a processor and a memory, and generates the driving information QJ.

The driving information generation unit 45 acquires the driving information QJ from various sensors disposed in the vehicle VC. The driving information QJ includes, for example, vehicle speed information, acceleration information, accelerator operation information, brake operation information, and steering wheel operation information.

Note that the driving information QJ may include road information about a road on which the vehicle VC travels. The road information includes, for example, construction information or traffic congestion information.

The vehicle speed information indicates a travel speed of the vehicle VC. The acceleration information indicates an acceleration detected by an acceleration sensor. The acceleration sensor detects, for example, an acceleration in an up-down direction, an acceleration in a front-rear direction, and an acceleration in a left-right direction of the vehicle VC.

The accelerator operation information indicates an operation state of an acceleration pedal caused by the user U. The accelerator operation information includes, for example, a pressed amount and a pressing speed of the acceleration pedal.

The brake operation information indicates an operation state of a brake pedal caused by the user U. The brake operation information includes, for example, a pressed amount and a pressing speed of the brake pedal.

The steering wheel operation information indicates an operation state of a steering wheel caused by the user U. The steering wheel operation information includes, for example, a steering angle and a change speed of the steering angle of the steering wheel.

The driving information generation unit 45 outputs the driving information QJ acquired from the various sensors to the ECU 41. The ECU 41 transmits the driving information QJ to the smartphone 1 via the fourth wireless communication interface 43.

The fourth position detection unit 46 includes a GNSS sensor. The fourth position detection unit 46 measures a position, that is, a latitude, a longitude, an altitude, and the like of the vehicle VC, by receiving a positioning signal transmitted from a GNSS satellite.

The fourth position detection unit 46 outputs position information indicating the measured position to the ECU 41.

### 4. Configuration of Smartphone

Next, a configuration of the smartphone 1 will be described with reference to FIG. 4. FIG. 4 is a diagram showing an example of the configuration of the smartphone 1.

The smartphone 1 includes a first control unit 11, a first clock unit 12, a first wireless communication interface 13, a first touch panel 14, a first position detection unit 15, a speaker 16, and a microphone 17.

The first control unit 11 controls each unit of the smartphone 1. The first control unit 11 includes a first processor 11A and a first memory 11B.

The first memory 11B consists of, for example, a non-volatile semiconductor memory, or volatile and non-volatile semiconductor memories. The first processor 11A consists of a CPU, an MPU, or the like. The first processor 11A may consist of a single processor or can also consist of a plurality of processors.

The first processor 11A controls each unit of the smartphone 1 by executing a first control program PG1 stored in the first memory 11B.

The first clock unit 12 has a clock function of measuring the date and time. The first clock unit 12 acquires the date and time information from the server apparatus (not illustrated). The first clock unit 12 adjusts the clock function by using the acquired date and time information. The first clock unit 12 displays the date and time information on the first touch panel 14.

The first wireless communication interface 13 includes an antenna that receives a wireless signal and an interface circuit that processes the wireless signal received by the antenna. The first wireless communication interface 13 performs wireless communication with external apparatuses including the server apparatus (not illustrated), the smartwatch 2, the information holding institution 3, and the in-vehicle apparatus 4.

The first touch panel 14 includes a display panel and a touch sensor. The display panel consists of a liquid crystal panel, an organic EL panel, or the like. The touch sensor detects a touch operation of the user U on the display panel.

The first touch panel 14 displays an image on the display panel in accordance with an instruction from the first control unit 11. The first touch panel 14 displays, for example, an image indicating advice information AD on the display panel in accordance with an instruction from the first control unit 11.

The first touch panel 14 detects a touch operation of the user U on the display panel.

The first position detection unit 15 includes a GNSS sensor. The first position detection unit 15 measures a position, that is, a latitude, a longitude, an altitude, and the like of the smartphone 1, by receiving a positioning signal transmitted from a GNSS satellite. The first position detection unit 15 outputs position information indicating the measured position to the first control unit 11.

The speaker 16 outputs various speech in accordance with an instruction from the first control unit 11. For example, the speaker 16 outputs speech indicating the advice information AD in accordance with an instruction from the first control unit 11.

The microphone 17 accepts an input of various speech in accordance with an instruction from the first control unit 11.

The microphone 17 may accept, for example, speech from the user U. The microphone 17 outputs the accepted speech to the first control unit 11.

The first control unit 11 functions as an acquisition unit 111, an evaluation unit 112, a mode determination unit 113, a report unit 114, a notification unit 115, and an information storage unit 116, through the first processor 11A executing the first control program PG1 stored in the first memory 11B. To be specific, the first processor 11A functions as the acquisition unit 111, the evaluation unit 112, the mode determination unit 113, the report unit 114, and the notification unit 115 by executing the first control program PG1 stored in the first memory 11B. The first processor 11A causes the first memory 11B to function as the information storage unit 116 by executing the first control program PG1 stored in the first memory 11B.

The first processor 11A corresponds to an example of the “processor”.

The first control program PG1 corresponds to an example of the “health state evaluation program”.

The information storage unit 116 stores the biological information BJ and the driving information QJ. The biological information BJ includes the first biological information BJ1 and the second biological information BJ2.

The first biological information BJ1 is, for example, acquired from the smartwatch 2 by the acquisition unit 111, and stored in the information storage unit 116 by the acquisition unit 111.

The second biological information BJ2 is, for example, acquired from the information holding institution 3 by the acquisition unit 111, and stored in the information storage unit 116 by the acquisition unit 111.

The driving information QJ is, for example, acquired from the in-vehicle apparatus 4 by the acquisition unit 111, and stored in the information storage unit 116 by the acquisition unit 111.

The acquisition unit 111 acquires the biological information BJ. The biological information BJ includes the first biological information BJ1 and the second biological information BJ2.

The acquisition unit 111 acquires, for example, the first biological information BJ1 from the smartwatch 2.

The first biological information BJ1 includes, for example, the sleep information BJ11, the exercise information BJ12, and the stress information BJ13.

The sleep information BJ11 includes, for example, sleep time information and information indicating sleep quality. The sleep time information indicates, for example, a sleep time per day of the user U. The information indicating sleep quality includes, for example, REM sleep time information and non-REM sleep time information per day of the user U.

The exercise information BJ12 includes, for example, step number information and exercise time information. The step number information indicates, for example, a cumulative value of the number of steps per day of the user U. The exercise time information indicates, for example, a cumulative value of an exercise time per day of the user U.

The stress information BJ13 includes heartbeat information and heartbeat fluctuation information. The heartbeat information indicates, for example, a heart rate per minute. The heartbeat fluctuation information indicates a state of a change in the heart rate.

The acquisition unit 111 acquires, for example, the second biological information BJ2 from the information holding institution 3.

The information holding institution 3 is an institution that holds the second biological information BJ2, such as a hospital, an insurance company, a municipality, or a caregiving company.

The second biological information BJ2 includes the health diagnosis information about the user U.

The health diagnosis information may, for example, include vision examination information, hearing examination information, blood examination information, urine examination information, electrocardiogram examination information, endoscopy examination information, ultrasound examination information, X-ray examination information, and gastroscopy examination information. The health diagnosis information may include computed tomography (CT) examination information and magnetic resonance imaging (MRI) examination information.

The health diagnosis information may, for example, include brain CT examination information, brain MRI examination information, and electroencephalography examination information through a brain checkup or the like. When the user U has a history of hospitalization, surgery, or the like, the health diagnosis information may, for example, include hospitalization surgery information. When the user U has an illness, the health diagnosis information may, for example, include illness information.

For example, the acquisition unit 111 acquires, as the second biological information BJ2, a rating RS of each of five abilities indicating a driving ability AB. The driving ability AB consists of, for example, a planning ability AB1, a perception ability AB2, an attention ability AB3, a judgment ability AB4, and an operation ability AB5.

The rating RS consists of the planning ability rating RS1, the perception ability rating RS2, the attention ability rating RS3, the judgment ability rating RS4, and the operation ability rating RS5.

The planning ability rating RS1 indicates the rating RS corresponding to the planning ability AB1. The planning ability AB1 is, for example, an ability of the user U to make a driving plan. The driving plan is, for example, a plan to cause the vehicle VC to travel from a parking position to a destination.

The perception ability rating RS2 indicates the rating RS corresponding to the perception ability AB2. The perception ability AB2 is, for example, an ability to perceive a surrounding situation while the user U is driving.

The attention ability rating RS3 indicates the rating RS corresponding to the attention ability AB3. The attention ability AB3 is, for example, an ability to pay attention to the surrounding situation in order to anticipate danger when the user U is driving.

The judgment ability rating RS4 indicates the rating RS corresponding to the judgment ability AB4. The judgment ability AB4 is, for example, an ability to judge whether to perform any type of operation in accordance with the surrounding situation while the user U is driving.

The operation ability rating RS5 indicates the rating RS corresponding to the operation ability AB5. The operation ability AB5 is, for example, an ability to perform an appropriate driving operation in accordance with the surrounding situation while the user U is driving.

The acquisition unit 111 outputs speech VQ to the user U at a predetermined timing set in advance, and acquires response speech VA being speech as a response from the user U to this speech.

The predetermined timing set in advance includes a timing when the user U enters the vehicle VC.

The predetermined timing set in advance may be, for example, ten minutes before the user U starts driving the vehicle VC and when a distance between the user U and the vehicle VC reaches 100 m while the user U is moving towards the vehicle VC in order to start driving.

The acquisition unit 111 outputs the speech VQ from the speaker 16. The speech VQ is, for example, speech for asking a question to the user U. The speech VQ is, for example, speech indicating “How are you?”

The acquisition unit 111 acquires the response speech VA from the user U to the speech VQ. The response speech VA is, for example, speech indicating “I'm good.” The response speech VA is, for example, speech indicating “I'm sleepy.”

The acquisition unit 111 acquires the driving information QJ and the first biological information BJ1 of a period from when the user U enters the vehicle VC until the user U gets out.

For example, the acquisition unit 111 acquires, from the in-vehicle apparatus 4, the driving information QJ of the period from when the user U enters the vehicle VC until the user U gets out. The acquisition unit 111 causes the information storage unit 116 to store the driving information QJ.

For example, the acquisition unit 111 acquires, from the smartwatch 2, the first biological information BJ1 of the period from when the user U enters the vehicle VC until the user U gets out. The acquisition unit 111 causes the information storage unit 116 to store the first biological information BJ1.

The evaluation unit 112 evaluates whether the health state of the user U is an appropriate state as the driver of the vehicle VC, by correcting the second biological information BJ2 using the first biological information BJ1.

For example, the evaluation unit 112 corrects, using the first biological information BJ1, each of the planning ability rating RS1, the perception ability rating RS2, the attention ability rating RS3, the judgment ability rating RS4, and the operation ability rating RS5 constituting the second biological information BJ2.

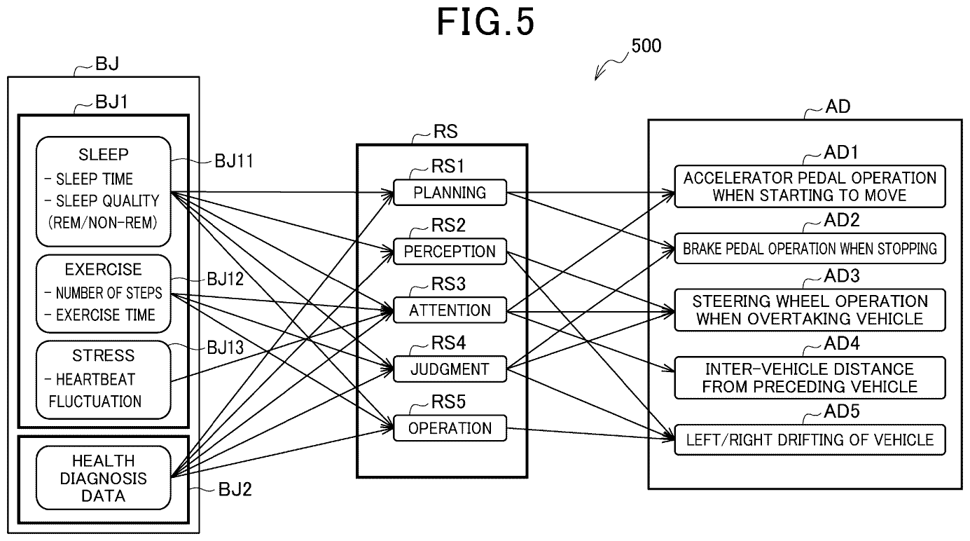

Correction processing by the evaluation unit 112 will be described further with reference to FIG. 5.

The evaluation unit 112 evaluates whether the health state of the user U is an appropriate state as the driver of the vehicle VC for each of the planning ability AB1, the perception ability AB2, the attention ability AB3, the judgment ability AB4, and the operation ability AB5.

The evaluation unit 112 evaluates, for example, how many points out of ten each of the planning ability rating RS1, the perception ability rating RS2, the attention ability rating RS3, the judgment ability rating RS4, and the operation ability rating RS5 has.

The evaluation unit 112 determines that the health state of the user U is an appropriate state, when each of the planning ability rating RS1, the perception ability judgment ability rating RS4, and the operation ability rating RS5 is, for example, equal to or higher than eight points.

The evaluation unit 112 determines that the health state of the user U is a state requiring attention, when each of the planning ability rating RS1, the perception ability rating RS2, the attention ability rating RS3, the judgment ability rating RS4, and the operation ability rating RS5 is, for example, equal to or higher than four points and equal to or lower than seven points.

The evaluation unit 112 determines that the health state of the user U is not an appropriate state, when each of the planning ability rating RS1, the perception ability rating RS2, the attention ability rating RS3, the judgment ability rating RS4, and the operation ability rating RS5 is, for example, equal to or lower than three points.

The report unit 114 determines a content of the advice information AD to be output to the user U while the user U is driving, based on the planning ability rating RS1, the perception ability rating RS2, the attention ability rating RS3, the judgment ability rating RS4, and the operation ability rating RS5.

The report unit 114 selects, for example, the advice information AD corresponding to a rating with the lowest score from among the planning ability rating RS1, the perception ability rating RS2, the attention ability rating RS3, the judgment ability rating RS4, and the operation ability rating RS5.

The advice information AD will be described further with reference to FIG. 5.

The mode determination unit 113 determines a mode EX of the advice information AD to be reported to the user U, based on the response speech VA acquired by the acquisition unit 111.

The mode determination unit 113 evaluates, for example, the health state and a stress level of the user U, based on an intonation, volume, and the like of the response speech VA. The mode determination unit 113 determines the mode EX of the advice information AD, in accordance with the health state and the stress level of the user U.

For example, the mode determination unit 113 determines, based on the response speech VA, to use a mode EX in which the user U is encouraged as the mode EX of the advice information AD, when it is evaluated that the user U is sleepy or lacks energy. The mode EX in which the user U is encouraged is, for example, a mode in which the advice information AD is reported using speech in a loud voice with a bright atmosphere.

For example, the mode determination unit 113 determines, based on the response speech VA, to use a normal mode as the mode EX of the advice information AD, when it is evaluated that the user U feels good.

For example, the mode determination unit 113 determines, based on the response speech VA, a mode in which the advice information AD is reported using speech in a low voice with a calm atmosphere as the mode EX of the advice information AD, when it is evaluated that the user U is experiencing stress.

The report unit 114 reports the advice information AD to the user U using the mode EX determined by the mode determination unit 113.

The report unit 114 outputs the speech indicating the advice information AD from, for example, the speaker 16. The report unit 114 displays an image indicating the advice information AD on, for example, the first touch panel 14.

The notification unit 115 notifies the information holding institution 3 of the driving information QJ and the first biological information BJ1 of the period from when the user U enters the vehicle VC until the user U gets out.

For example, when the user U gets out, the notification unit 115 reads the driving information QJ and the first biological information BJ1 stored in the information storage unit 116, and notifies the information holding institution 3 of the driving information QJ and the first biological information BJ1.

The acquisition unit 111 acquires advice information AJ corresponding to the driving information QJ and the first biological information BJ1 from the information holding institution 3. The advice information AJ indicates an address for the user U input by a specialist such as a physician that belongs to the information holding institution 3.

When the acquisition unit 111 acquires the advice information AJ, the report unit 114 outputs speech indicating the advice information AJ from the speaker 16. When the acquisition unit 111 acquires the advice information AJ, the report unit 114 displays an image indicating the advice information AJ on the first touch panel 14.

### 5. Correlation Between Biological Information, Rating, and Advice Information

Next, a correlative relationship between the biological information BJ, the rating RS, and the advice information AD will be described with reference to FIG. 5. FIG. 5 is a diagram showing an example of a correlation between the biological information BJ, the rating RS, and the advice information AD. The rating RS corresponds to each of the five abilities indicating the driving ability AB. FIG. 5 illustrates a correlation diagram 500.

The correlation diagram 500 includes the biological information BJ, the rating RS, and the advice information AD.

As shown in FIG. 5, the biological information BJ includes the first biological information BJ1 and the second biological information BJ2.

The first biological information BJ1 includes, for example, the sleep information BJ11, the exercise information BJ12, and the stress information BJ13.

The second biological information BJ2 includes the health diagnosis information about the user U. The second biological information BJ2 includes the planning ability rating RS1, the perception ability rating RS2, the attention ability rating RS3, the judgment ability rating RS4, and the operation ability rating RS5.

The rating RS includes the planning ability rating RS1, the perception ability rating RS2, the attention ability rating RS3, the judgment ability rating RS4, and the operation ability rating RS5.

For example, the evaluation unit 112 corrects, using the first biological information BJ1, each of the planning ability rating RS1, the perception ability judgment ability rating RS4, and the operation ability rating RS5 constituting the second biological information BJ2.

In other words, the evaluation unit 112 determines the planning ability rating RS1 by correcting, using the first biological information BJ1, the planning ability rating RS1 constituting the second biological information BJ2. The evaluation unit 112 determines the perception ability rating RS2 by correcting, using the first biological information BJ1, the perception ability rating RS2 constituting the second biological information BJ2. The evaluation unit 112 determines the attention ability rating RS3 by correcting, using the first biological information BJ1, the attention ability rating RS3 constituting the second biological information BJ2. The evaluation unit 112 determines the judgment ability rating RS4 by correcting, using the first biological information BJ1, the judgment ability rating RS4 constituting the second biological information BJ2. The evaluation unit 112 determines the operation ability rating RS5 by correcting, using the first biological information BJ1, the operation ability rating RS5 constituting the second biological information BJ2.

Arrows between the first biological information BJ1 and the rating RS in FIG. 5 indicate correlations.

For example, the sleep information BJ11 of the first biological information BJ1 correlates with the planning ability rating RS1, the perception ability rating RS2, the attention ability rating RS3, the judgment ability rating RS4, and the operation ability rating RS5 of the rating RS. Accordingly, the evaluation unit 112 corrects, using the sleep information BJ11 of the first biological information BJ1, each of the planning ability rating RS1, the perception ability rating RS2, the attention ability rating RS3, the judgment ability rating RS4, and the operation ability rating RS5 constituting the second biological information BJ2.

The exercise information BJ12 of the first biological information BJ1 correlates with the attention ability rating RS3, the judgment ability rating RS4, and the operation ability rating RS5 of the rating RS. Accordingly, the evaluation unit 112 corrects, using the exercise information BJ12 of the first biological information BJ1, each of the attention ability rating RS3, the judgment ability rating RS4, and the operation ability rating RS5 constituting the second biological information BJ2.

The stress information BJ13 of the first biological information BJ1 correlates with the attention ability rating RS3 of the rating RS. Accordingly, the evaluation unit 112 corrects, using the stress information BJ13 of the first biological information BJ1, the attention ability rating RS3 constituting the second biological information BJ2.

In other words, the evaluation unit 112 determines the planning ability rating RS1 by correcting, using the sleep information BJ11 of the first biological information BJ1, the planning ability rating RS1 constituting the second biological information BJ2.

The evaluation unit 112 determines the perception ability rating RS2 by correcting, using the sleep information BJ11 of the first biological information BJ1, the perception ability rating RS2 constituting the second biological information BJ2.

The evaluation unit 112 determines the attention ability rating RS3 by correcting, using the sleep information BJ11, the exercise information BJ12 and the stress information BJ13 of the first biological information BJ1, the attention ability rating RS3 constituting the second biological information BJ2.

The evaluation unit 112 determines the judgment ability rating RS4 by correcting, using the sleep information BJ11 and the exercise information BJ12 of the first biological information BJ1, the judgment ability rating RS4 constituting the second biological information BJ2.

The evaluation unit 112 determines the operation ability rating RS5 by correcting, using the sleep information BJ11 and the exercise information BJ12 of the first biological information BJ1, the operation ability rating RS5 constituting the second biological information BJ2.

As shown in FIG. 5, the advice information AD includes, for example, the first advice information AD1, the second advice information AD2, the third advice information AD3, the fourth advice information AD4, and the fifth advice information AD5.

The first advice information AD1 is, for example, the advice information AD relating to an operation of the acceleration pedal when the user U starts moving the vehicle VC. The first advice information AD1 is, for example, the advice information AD encouraging not to abruptly start moving.

The second advice information AD2 is, for example, the advice information AD relating to an operation of the brake pedal when the user U stops the vehicle VC. The second advice information AD2 is, for example, the advice information AD encouraging not to abruptly stop.

The third advice information AD3 is, for example, the advice information AD relating to an operation of the steering wheel when the user U causes the vehicle VC to overtake another vehicle. The third advice information AD3 is, for example, the advice information AD encouraging not to abruptly steer.

The fourth advice information AD4 is, for example, the advice information AD relating to an inter-vehicle distance between the vehicle VC that the user U is driving and another vehicle traveling in front. The fourth advice information AD4 is, for example, the advice information AD encouraging to keep an appropriate inter-vehicle distance.

The fifth advice information AD5 is the advice information AD relating to drifting in the left-right direction of the vehicle VC that the user U is driving. The fifth advice information AD5 is, for example, the advice information AD encouraging to suppress drifting in the left-right direction of the vehicle VC.

Arrows between the rating RS and the advice information AD in FIG. 5 indicate correlations.

The report unit 114 selects, for example, the advice information AD corresponding to the rating with the lowest score from among the planning ability rating RS1, the perception ability rating RS2, the attention ability rating RS3, the judgment ability rating RS4, and the operation ability rating RS5.

For example, as shown in FIG. 5, the planning ability rating RS1 correlates with the first advice information AD1 and the second advice information AD2. Accordingly, the report unit 114 reports, for example, the first advice information AD1 and the second advice information AD2 with priority, when the planning ability rating RS1 is the rating with the lowest score.

The perception ability rating RS2 correlates with the third advice information AD3 and the fifth advice information AD5. Accordingly, the report unit 114 reports, for example, the third advice information AD3 and the fifth advice information AD5 with priority, when the perception ability rating RS2 is the rating with the lowest score.

The attention ability rating RS3 correlates with the first advice information AD1, the third advice information AD3, and the fourth advice information AD4. Accordingly, the report unit 114 reports, for example, the first advice information AD1, the third advice information AD3, and the fourth advice information AD4 with priority, when the attention ability rating RS3 is the rating with the lowest score.

The judgment ability rating RS4 correlates with the second advice information AD2, the third advice information AD3, and the fifth advice information AD5. Accordingly, the report unit 114 reports, for example, the second advice information AD2, the third advice information AD3, and the fifth advice information AD5 with priority, when the judgment ability rating RS4 is the rating with the lowest score.

The operation ability rating RS5 correlates with the fifth advice information AD5. Accordingly, the report unit 114 reports, for example, the fifth advice information AD5 with priority, when the operation ability rating RS5 is the rating with the lowest score.

### 6. Processing of Smartphone

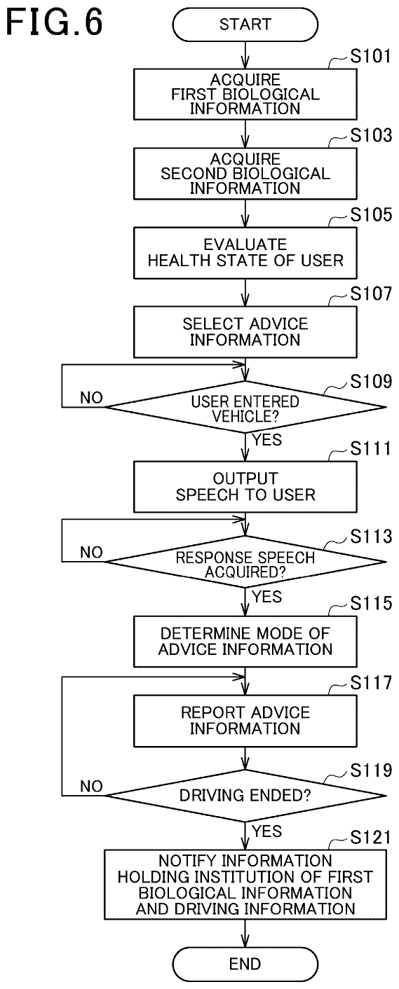

Next, processing of the smartphone 1 will be described with reference to FIG. 6. FIG. 6 is a flowchart showing an example of the processing of the smartphone 1.

First, in step S101, the acquisition unit 111 acquires the first biological information BJ1 from the smartwatch 2. The first biological information BJ1 includes, for example, the sleep information BJ11, the exercise information BJ12, and the stress information BJ13.

Next, in step S103, the acquisition unit 111 acquires the second biological information BJ2 from the information holding institution 3. The second biological information BJ2 includes the health diagnosis information about the user U.

Next, in step S105, the evaluation unit 112 evaluates whether the health state of the user U is an appropriate state as the driver of the vehicle VC, by correcting the second biological information BJ2 using the first biological information BJ1. For example, the evaluation unit 112 corrects, using the first biological information BJ1, each of the planning ability rating RS1, the perception ability rating RS2, the attention ability rating RS3, the judgment ability rating RS4, and the operation ability rating RS5 constituting the second biological information BJ2.

Next, in step S107, the report unit 114 determines a content of the advice information AD to be output to the user U while the user U is driving, based on the planning ability rating RS1, the perception ability rating RS2, the attention ability rating RS3, the judgment ability rating RS4, and the operation ability rating RS5.

Next, in step S109, the acquisition unit 111 determines whether the user U has entered the vehicle VC.

When the acquisition unit 111 determines that the user U has not entered the vehicle VC (NO in step S109), the processing enters a standby state. When the acquisition unit 111 determines that the user U has entered the vehicle VC (YES in step S109), the processing proceeds to step S111.

Then, in step S111, the acquisition unit 111 outputs the speech VQ for asking a question to the user from the speaker 16.

Next, in step S113, the acquisition unit 111 determines whether the response speech VA from the user U to the speech VQ has been acquired.

When the acquisition unit 111 determines that the response speech VA from the user U to the speech VQ has not been acquired (NO in step S113), the processing enters a standby state. When the acquisition unit 111 determines that the response speech VA from the user U to the speech VQ has been acquired (YES in step S113), the processing proceeds to step S115.

Then, in step S115, the mode determination unit 113 determines the mode EX of the advice information AD to be reported to the user U, based on the response speech VA.

Next, in step S117, the report unit 114 reports the advice information AD determined in step S107 to the user U using the mode EX determined by the mode determination unit 113.

Next, in step S119, the notification unit 115 determines whether the user U has stopped driving the vehicle VC.

When the notification unit 115 determines that the user U has not stopped driving the vehicle VC (NO in step S119), the processing returns to step S117. When the notification unit 115 determines that the user U has stopped driving the vehicle VC (YES in step S119), the processing proceeds to step S121.

Then, in step S121, the notification unit 115 notifies the information holding institution 3 of the driving information QJ and the first biological information BJ1 of the period from when the user U enters the vehicle VC until the user U gets out. Subsequently, the processing ends.

Steps S101 and S103 correspond to an example of an “acquisition step”.

Step S105 corresponds to an example of an “evaluation step”.

### 7. Configurations and Effects

As described with reference to FIGS. 1 to 6, the smartphone 1 according to the present embodiment includes: the acquisition unit 111 configured to acquire the first biological information BJ1 from the smartwatch 2 that is worn by the user U and detects the biological information about the user U, and acquire the second biological information BJ2 from the information holding institution 3 that holds the biological information about the user U; and the evaluation unit 112 configured to evaluate whether the health state of the user U is an appropriate state as the driver of the vehicle VC, based on the first biological information BJ1 and the second biological information BJ2.

Therefore, it is possible to appropriately evaluate whether the health state related to the driving ability of the user U, since it is evaluated whether the health state of the user U is an appropriate state as the driver of the vehicle VC based on the first biological information BJ1 and the second biological information BJ2.

In the smartphone 1, the evaluation unit 112 evaluates whether the health state of the user U is the appropriate state, by correcting the second biological information BJ2 using the first biological information BJ1.

Therefore, it is possible to appropriately evaluate the health state related to the driving ability of the user U, since it is evaluated whether the health state of the user U is an appropriate state as the driver of the vehicle VC, by correcting the second biological information BJ2 using the first biological information BJ1. For example, it is possible to appropriately evaluate the health state related to the driving ability of the user U, by correcting, using the first biological information BJ1 indicating daily physical condition changes, the second biological information BJ2 being a result of precise examination, when the second biological information BJ2 is the health diagnosis information.

In the smartphone 1, the evaluation unit 112 evaluates whether the health state of the user U is the appropriate state as for each of the planning ability AB1, the perception ability AB2, the attention ability AB3, the judgment ability AB4, and the operation ability AB5, and the report unit 114 is provided that reports the advice information AD to the user U based on an evaluation result from the evaluation unit 112.

Therefore, it is possible to appropriately evaluate the health state related to the driving ability of the user U, since the health state of the user U is evaluated for each of the planning ability AB1, the perception ability AB2, the attention ability AB3, the judgment ability AB4, and the operation ability AB5.

In the smartphone 1, the report unit 114 is provided that reports the advice information AD to the user U, based on the evaluation result from the evaluation unit 112.

Therefore, it is possible to report appropriate advice information AD to the user U, since the advice information AD is reported to the user U based on the evaluation result from the evaluation unit 112.

In the smartphone 1, the acquisition unit 111 outputs the speech VQ to the user U at the predetermined timing set in advance, and acquires the response speech VA being the speech as a response from the user U to the speech VQ, and the mode determination unit 113 is provided that determines the mode EX of the advice information AD based on the response speech VA.

Therefore, it is possible to appropriately determine the mode EX of the advice information AD, since the mode EX of the advice information AD is determined based on the response speech VA.

In the smartphone 1, the predetermined timing includes the timing when the user U enters the vehicle VC, the acquisition unit 111 outputs the speech VQ to the user U and acquires the response speech VA when the user U enters the vehicle VC, and the mode determination unit 113 determines the mode EX of the advice information AD based on the response speech VA.

Therefore, it is possible to appropriately determine the mode EX of the advice information AD, since the mode EX of the advice information AD is determined based on the response speech VA from when the user U enters the vehicle VC.

In the smartphone 1, the acquisition unit 111 acquires the driving information QJ and the first biological information BJ1 of the period from when the user U enters the vehicle VC until the user U gets out, and a notification unit 115 is provided that notifies the information holding institution 3 of the driving information QJ and the first biological information BJ1.

Therefore, it is possible to provide the information holding institution 3 with valuable information while recognizing the health state of the user U, since the information holding institution 3 is notified of the driving information QJ and the first biological information BJ1 of the period from when the user U enters the vehicle VC until the user U gets out.

In the smartphone 1, the acquisition unit 111 acquires the advice information AJ for the user U from a specialist of the information holding institution 3, based on the driving information QJ and the first biological information BJ1, and the report unit 114 is provided that reports the advice information AJ to the user U.

Thus, it is possible to provide useful information to the user U, since the advice information AJ for the user U from a specialist of the information holding institution 3 is acquired and the advice information AJ is reported to the user U. Therefore, it is possible the improve user-friendliness for the user U.

A health state evaluation method according to the present embodiment includes: the acquisition step of acquiring the first biological information BJ1 from the smartwatch 2 that is worn by the user U and detects the biological information about the user U, and acquiring the second biological information BJ2 from the information holding institution 3 that holds the biological information about the user U; and the evaluation step of evaluating whether the health state of the user U is an appropriate state as the driver of the vehicle VC, based on the first biological information BJ1 and the second biological information BJ2.

Thus, the health state evaluation method according to the present embodiment exhibits operations and effects similar to those of the smartphone 1 according to the present embodiment.

The first control program PG1 according to the present embodiment causes the first processor 11A included in the smartphone 1 to function as: the acquisition unit 111 configured to acquire the first biological information BJ1 from the smartwatch 2 that is worn by the user U and detects the biological information about the user U, and acquire the second biological information BJ2 from the information holding institution 3 that holds the biological information about the user U; and the evaluation unit 112 configured to evaluate whether the health state of the user U is an appropriate state as the driver of the vehicle VC, based on the first biological information BJ1 and the second biological information BJ2.

Thus, the first control program PG1 according to the present embodiment exhibits operations and effects similar to those of the smartphone 1 according to the present embodiment.

### 8. Other Embodiments

The embodiment described above is a preferred embodiment of the present invention. However, the present invention is not limited to the embodiment described above, and can be implemented with a variety of modifications within the scope or the spirit of the present invention.

In the present embodiment, a case is described in which the “health state evaluation apparatus” is implemented as the smartphone 1, but the embodiment is not limited thereto. The “health state evaluation apparatus” may, for example, be implemented as a personal computer, a tablet apparatus, a server apparatus, or the like.

In the present embodiment, a case is described in which the “device” is implemented as the smartwatch 2, but the embodiment is not limited thereto. The “device” may be worn by the user U and may generate the first biological information BJ1.

For example, the “device” may be worn on an arm, the head, a leg, or the like of the user U. For example, the “device” may be smart innerwear attached to innerwear worn by the user U. For example, the “device” may be a sensing device attached to the skin, inside the body, or the like of the user U.

In the present embodiment, a case is described in which the report unit 114 outputs speech indicating the advice information AD from the speaker 16, but the embodiment is not limited thereto. The report unit 114 may output the speech indicating the advice information AD from a speaker connected to the in-vehicle apparatus 4 and installed in the vehicle VC.

In the present embodiment, a case is described in which the report unit 114 displays an image indicating the advice information AD on the first touch panel 14, but the embodiment is not limited thereto. The report unit 114 may display the image indicating the advice information AD on the fourth touch panel 44 included in the in-vehicle apparatus 4.

For example, each of the configurations of the smartwatch 2 shown in FIG. 2, the in-vehicle apparatus 4 shown in FIG. 3, and the smartphone 1 shown in FIG. 4 indicates a functional configuration, and the specific embodiment thereof is not particularly limited. In other words, hardware individually corresponding to the respective functional units does not necessarily need to be implemented, and it is possible to adopt a configuration in which one processor executes a program to realize functions of a plurality of functional units. Part of functions realized by software in the above embodiments may be realized by hardware or part of functions realized by hardware may be realized by software.

The units of processing of the flowchart shown in FIG. 6 are divided in accordance with the main processing content in order to facilitate understanding of the processing of the smartphone 1, but the present invention is not limited by the division method, naming, or the like of the units of processing shown in the flowchart of FIG. 6. The processing of the smartphone 1 can be further divided into more units of processing in accordance with the content of the processing, or one unit of processing can be further divided to include more processing. The processing order of the above flowchart is also not limited to the illustrated example.

### 9. Configurations Supported by the Above Embodiments

The above embodiments are specific examples of the following configurations.

### Configuration 1

A health state evaluation apparatus includes: an acquisition unit configured to acquire first biological information from a device that is worn by a user and detects biological information about the user, and acquire second biological information from an information holding institution that holds biological information about the user; and an evaluation unit configured to evaluate whether a health state of the user is an appropriate state as a driver of a vehicle, based on the first biological information and the second biological information.

The health state evaluation apparatus of Configuration 1 can appropriately evaluate the health state related to the driving ability of the user, since it is evaluated whether the health state of the user is an appropriate state as the driver of the vehicle based on the first biological information and the second biological information.

### Configuration 2

The health state evaluation apparatus according to Configuration 1, in which the evaluation unit evaluates whether the health state of the user is the appropriate state, by correcting the second biological information using the first biological information.

The health state evaluation apparatus of Configuration 2 can appropriately evaluate the health state related to the driving ability of the user, since it is evaluated whether the health state of the user is an appropriate state as the driver of the vehicle, by correcting the second biological information using the first biological information. For example, it is possible to appropriately evaluate the health state related to the driving ability of the user, by correcting, using the first biological information indicating daily physical condition changes, the second biological information being the result of precise examination, when the second biological information is the health diagnosis information.

### Configuration 3

The health state evaluation apparatus according to Configuration 1 or 2, in which the evaluation unit evaluates whether the health state of the user is the appropriate state for each of a planning ability, a perception ability, an attention ability, a judgment ability, and an operation ability.

The health state evaluation apparatus of Configuration 3 can appropriately evaluate the health state related to the driving ability of the user, since the health state of the user is evaluated for each of the planning ability, the perception ability, the attention ability, the judgment ability, and the operation ability.

### Configuration 4

The health state evaluation apparatus according to any one of Configurations 1 to 3 includes a report unit configured to report advice information to the user, based on an evaluation result from the evaluation unit.

The health state evaluation apparatus of Configuration 4 can report appropriate advice information to the user, since the advice information is reported to the user based on the evaluation result from the evaluation unit.

### Configuration 5

The health state evaluation apparatus according to any one of Configurations 1 to 4, in which the acquisition unit outputs speech to the user at a predetermined timing set in advance, and acquires response speech being speech as a response from the user to the speech, and the health state evaluation apparatus includes a mode determination unit configured to determine a mode of the advice information, based on the response speech.

The health state evaluation apparatus of Configuration 5 can appropriately determine the mode of the advice information, since the mode of the advice information is determined based on the response speech.

### Configuration 6

The health state evaluation apparatus according to Configuration 5, in which the predetermined timing includes a timing when the user enters the vehicle, the acquisition unit outputs the speech to the user and acquires the response speech, when the user enters the vehicle, and the mode determination unit determines the mode of the advice information, based on the response speech.

The health state evaluation apparatus of Configuration 6 can appropriately determine the mode of the advice information, since the mode of the advice information is determined based on the response speech from when the user enters the vehicle.

### Configuration 7

The health state evaluation apparatus according to any one of Configurations 1 to 6, in which the acquisition unit acquires the driving information and the first biological information of a period from when the user enters the vehicle until the user gets out, and the health state evaluation apparatus includes a notification unit configured to notify the information holding institution of the driving information and the first biological information.

The health state evaluation apparatus of Configuration 7 can provide the information holding institution with valuable information while recognizing the health state of the user, since the information holding institution is notified of the driving information and the first biological information of the period from when the user enters the vehicle until the user gets out.

### Configuration 8

The health state evaluation apparatus according to Configuration 7, in which the acquisition unit acquires, from the information holding institution, advice information for the user from a specialist, based on the driving information and the first biological information, and the health state evaluation apparatus includes a report unit configured to report the advice information to the user.

The health state evaluation apparatus of Configuration 8 can provide useful information to the user, since the advice information for the user from a specialist of the information holding institution is acquired and the advice information is reported to the user. Therefore, it is possible the improve user-friendliness for the user.

### Configuration 9

A health state evaluation method includes: an acquisition step of acquiring first biological information from a device that is worn by a user and detects biological information about the user, and acquiring second biological information from an information holding institution that holds biological information about the user; and an evaluation step of evaluating whether a health state of the user is an appropriate state as a driver of a vehicle, based on the first biological information and the second biological information.

The health state evaluation method of Configuration 9 has a configuration similar to that of the health state evaluation apparatus of Configuration 1, and thus exhibits operations and effects similar to those of the health state evaluation apparatus of Configuration 1.

### Configuration 10

A non-transitory computer readable storage medium storing a health state evaluation program causes a processor included in a health state evaluation apparatus to function as: an acquisition unit configured to acquire first biological information from a device that is worn by a user and detects biological information about the user, and acquire second biological information from an information holding institution that holds biological information about the user; and an evaluation unit configured to evaluate whether a health state of the user is an appropriate state as a driver of a vehicle, based on the first biological information and the second biological information.

The storage medium of Configuration 10 has a configuration similar to that of the health state evaluation apparatus of Configuration 1, and thus exhibits operations and effects similar to those of the health state evaluation apparatus of Configuration 1.

### REFERENCE SIGNS LIST

100 health state evaluation system
1 smartphone (health state evaluation apparatus)
11 first control unit
11A first processor (processor)
11B first memory
111 acquisition unit
112 evaluation unit
113 mode determination unit
114 report unit
115 notification unit
116 information storage unit
16 speaker
17 microphone
14 first touch panel
2 smartwatch (device)
25 biological information generation unit
3 information holding institution
4 in-vehicle apparatus
41 ECU
45 driving information generation unit
AB driving ability
AB1 planning ability
AB2 perception ability
AB3 attention ability
AB4 judgment ability
AB5 operation ability
AD advice information
AJ advice information
BJ biological information
BJ1 first biological information
BJ11 sleep information
BJ12 exercise information
BJ13 stress information
BJ2 second biological information
EX mode
PG1 first control program (health state evaluation program)
QJ driving information
RS rating
RS1 planning ability rating
RS2 perception ability rating
RS3 attention ability rating
RS4 judgment ability rating
RS5 operation ability rating
U user
VA response speech
VC vehicle
VQ speech

1. A health state evaluation apparatus, comprising:
an acquisition unit configured to acquire first biological information from a device that is worn by a user and detects biological information about the user, and acquire second biological information from an information holding institution that holds biological information about the user; and
an evaluation unit configured to evaluate whether a health state of the user is an appropriate state as a driver of a vehicle, based on the first biological information and the second biological information.

2. The health state evaluation apparatus according to claim 1, wherein the evaluation unit evaluates whether the health state of the user is the appropriate state, by correcting the second biological information using the first biological information.

3. The health state evaluation apparatus according to claim 1, wherein the evaluation unit evaluates whether the health state of the user is the appropriate state for each of a planning ability, a perception ability, an attention ability, a judgment ability, and an operation ability.

4. The health state evaluation apparatus according to claim 1, comprising a report unit configured to report advice information to the user, based on an evaluation result from the evaluation unit.

5. The health state evaluation apparatus according to claim 4, wherein
the acquisition unit outputs speech to the user at a predetermined timing set in advance, and acquires response speech being speech as a response from the user to the speech, and
the health state evaluation apparatus comprises a mode determination unit configured to determine a mode of the advice information, based on the response speech.

6. The health state evaluation apparatus according to claim 5, wherein
the predetermined timing includes a timing when the user enters the vehicle,
the acquisition unit outputs the speech to the user and acquires the response speech, when the user enters the vehicle, and
the mode determination unit determines the mode of the advice information, based on the response speech.

7. The health state evaluation apparatus according to claim 1, wherein
the acquisition unit acquires the driving information and the first biological information of a period from when the user enters the vehicle until the user gets out, and
the health state evaluation apparatus comprises a notification unit configured to notify the information holding institution of the driving information and the first biological information.

8. The health state evaluation apparatus according to claim 7, wherein
the acquisition unit acquires, from the information holding institution, advice information for the user from a specialist, based on the driving information and the first biological information, and
the health state evaluation apparatus comprises a report unit configured to report the advice information to the user.

9. A health state evaluation method, comprising:
an acquisition step of acquiring first biological information from a device that is worn by a user and detects biological information about the user, and acquiring second biological information from an information holding institution that holds biological information about the user; and
an evaluation step of evaluating whether a health state of the user is an appropriate state as a driver of a vehicle, based on the first biological information and the second biological information.

10. A non-transitory computer readable storage medium storing a health state evaluation program for causing a processor included in a health state evaluation apparatus to function as:
an acquisition unit configured to acquire first biological information from a device that is worn by a user and detects biological information about the user, and acquire second biological information from an information holding institution that holds biological information about the user; and
an evaluation unit configured to evaluate whether a health state of the user is an appropriate state as a driver of a vehicle, based on the first biological information and the second biological information.
